(19) United States
(12) Patent Application Publication
(10) Pub. No.: US 2023/0090912 A1
(43) Pub. Date: Mar. 23, 2023

(54) METHODS OF CONTROLLING ANTIBODY HETEROGENEITY

(71) Applicant: Regeneron Pharmaceuticals, Inc., Tarrytown, NY (US)

(72) Inventors: Philip Mellors, Limerick (IE); John Hourihan, Limerick (IE); John Crowley, Limerick (IE)

(21) Appl. No.: 17/948,382

(22) Filed: Sep. 20, 2022

Related U.S. Application Data
(60) Provisional application No. 63/246,047, filed on Sep. 20, 2021.

(51) Int. Cl. C12N 5/075; C07K 16/28

(52) U.S. Cl. CPC C12N 5/0609; C07K 16/2866; C07K 16/2896; C07K 2317/20; C12N 2500/02

(57) ABSTRACT

The present inventions provide methods to control the heterogeneity of Fc-containing proteins, such as antibodies produced in cell culture, particularly mammalian cell culture by controlling culture pCO₂, as well as products produced by these methods. Among other things, the inventions provide for lowering the percentage of acidic charge variants in antibody products. Proteins that comprise Fc moieties include but are not limited to Fc-containing proteins, such as antibodies and antibody derivatives, and fragments of both.

The application claims priority to U.S. Application Ser. No. 63/246,047, filed Sep. 20, 2021, which is hereby incorporated by reference.

### FIELD OF THE INVENTIONS

The present inventions provide methods to control the heterogeneity of Fc-containing proteins produced in cell culture, particularly mammalian cell culture, as well as protein products and proteins produced by these methods. Proteins that comprise Fc moieties include Fc-containing proteins, such as antibodies.

### BACKGROUND OF THE INVENTIONS

Production of Fc-containing proteins, such as antibodies, in cell culture can result in charge variants, which come in two types referred to as acidic variants and basic variants. In addition, there is a main peak form. Fc refers to “fragment crystallizable,” which is the constant region found in antibody heavy chains as found in nature, and also is included in monoclonal antibodies, for example.

Acidic variants typically are more prevalent than basic variants in antibodies, and can result in deamidation, sialylation, glycation and fragmentation, which alters the stability, activity and potency of proteins that comprise Fc moieties (portions from the fragment crystallizable region of antibodies). Sissolak et al., J. Indust. Microbiol. Biotech. 46: 1167-78 (2019). Basic variants can cause increased binding of antibodies to Fc receptors. Hintersteiner et al., MABS 8: 1458-60 (2016).

Fc glycans also plays a role in safety, bioactivity, pharmacodynamics and pharmacokinetics. Reusch and Tejada, Glycobiol. 25: 1325-34 (2015). A phenomenon that can occur is known as non-glycosylated heavy chain (NGHC). NGHC variation can alter effector functions, such as opsonization. Opsonization concerns the Fc portions that are involved in ADCC (antibody-dependent cellular cytotoxicity), ADCP (antibody-dependent cellular phagocytosis) and CDC (complement-dependent cytotoxicity). NGHC variation can be a concern in some contexts (depending on disease state, administration route and type of Fc-containing protein), and be of lesser importance in others.

Thus, there exists the need to control charge variation and/or NGHC in proteins that comprise Fc moieties. This, however, can create situations where optimization of one can, but not always, lead to a possibly less favorable state for the other, as discussed in greater detail below. Due to the effects of acidic charge variants in antibodies, there usually is a desire to lessen the occurrence of such variants. Ultimately, charge variation can be a concern in some contexts (depending on disease state, administration route and type of Fc-containing protein), and be of lesser importance in others. The inventions described below address this need and other needs.

### SUMMARY OF THE INVENTIONS

The present inventions provide methods of controlling heterogeneity in Fc-containing proteins, such as antibodies, produced by mammalian cells in culture. The methods can comprise seeding media with mammalian cells that produce Fc-containing proteins; and culturing the cells under pCO₂ conditions that allow the mammalian cells to produce Fc-containing proteins. Preferably, CO₂ sparging is used to increase pCO₂ in the culture. Another approach is to allow pCO₂ to build up and be controlled with air sparging. Pressure reduction in bioreactors can also be used to control pCO₂. Combinations of CO₂ sparging, air/nitrogen sparging and pressure reduction can be employed. Charge variants are mainly due to alterations in the Fc region.

Depending on the objectives of the skilled person, combinations of CO₂ sparging, air/nitrogen sparging and pressure reduction can be employed in view of the teachings contained herein.

The inventions also provide methods for controlling, preferably reducing, the percentage of acidic charge variants in Fc-containing protein products, such as antibodies, produced by mammalian cells in culture, wherein the method comprises seeding media with mammalian cells that produce Fc-containing proteins; and culturing the cells under pCO₂ conditions that allow the mammalian cells to produce Fc-containing protein products with less acidic acid variants than would be obtained without the pCO₂ conditions, wherein the pCO₂ conditions are, for example, 120 mmHg to 140 mmHg of CO₂ in the media or as otherwise disclosed herein. The pCO₂ conditions can be attained by sparging, such as CO₂ sparging. Charge variants can be caused by alterations in the Fc region. The Fc-containing proteins produced under the pCO₂ conditions can have 0.5% to 4% less acidic variants than would be obtained without the pCO₂ conditions, for example. The Fc-containing proteins can be antibodies, such as antibodies that are capable of binding PD-1 factor or IL-4 receptors. Preferably, the antibodies are human monoclonal antibodies, preferably IgG antibodies, including subclasses such as IgG1 and IgG4. The mammalian cells are can be CHO, BHK, HEK293, HeLa, Human Amniotic, Per.C6 and Sp2/0 cells, for example. Cells can be culture under various pCO₂ conditions disclosed herein for 10-15 days, preferably about 14 days.

The inventions further provide methods that comprise seeding media with mammalian cells that produce Fc-containing proteins, such as antibodies; and culturing the cells under pCO₂ conditions that allow the mammalian cells to produce Fc-containing proteins, wherein the main peak form of Fc-containing proteins produced by the cells comprises between about 38% to about 65% of total Fc-containing proteins, the acidic variant of the Fc-containing proteins comprises about 20% to about 47% of total Fc-containing proteins and the basic variant of the Fc-containing proteins comprises up to about 36% of total Fc-containing proteins, which can be antibodies, derivatives and fragments of both. The cells can be cultured for about 10-15 days, preferably about 14 days. The pCO₂ conditions can be between about 30 mmHg and about 210 mmHg, 50 mmHg to 200 mmHg, 60 mmHg to 190 mmHg, 70 mmHg to 180 mmHg, 80 mmHg to 170 mmHg, 90 mmHg to 160 mmHg, 100 mmHg to 150 mmHg, 110 mmHg to 140 mmHg, 120 mmHg to 140 mmHg, 120 mmHg to 130 mmHg or any value within these ranges during the culturing, which is preferably maintained by CO₂ sparging, and can be measured using a CO₂ electrode. The cells can be any suitable mammalian cell, including CHO, BHK, HEK293, HeLa, Human Amniotic, Per.C6 and Sp2/0 cells.

The Fc-containing proteins can be antibodies, such as antibodies capable of binding PD-1 factor or IL-4 receptors. Preferably, the antibodies are human monoclonal antibodies, preferably all IgG antibodies, including subclasses such as IgG1 and IgG4.

The inventions also provide methods of controlling heterogeneity in antibodies, antibody derivatives or antibody fragments produced by mammalian cells in culture by seeding media with mammalian cells that produce antibodies, antibody derivatives or antibody fragments; and culturing the cells under pCO₂ conditions that allow the mammalian cells to produce antibodies, antibody derivatives or antibody fragments, wherein the main peak form of antibodies, antibody derivatives or antibody fragments produced by the cells comprise between about 50% to about 70% of total antibodies, antibody derivatives or antibody fragments, the acidic variant of the antibodies, antibody derivatives or antibody fragments comprise about 20% to about 47% of total antibodies, antibody derivatives or antibody fragments and the basic variant of the antibodies, antibody derivatives or antibody fragments comprise up to about 15% of total antibodies, antibody derivatives or antibody fragments. The basic variant of the antibodies, antibody derivatives or antibody fragments can comprise up to about 6%, about 8% or about 10%, and preferably no more than about 15% of total antibodies, antibody derivatives or antibody fragments. The main peak form of antibodies, antibody derivatives or antibody fragments produced by the cells can comprise between about 50% to about 65% of total antibodies, antibody derivatives or antibody fragments and the acidic variant of the antibodies, antibody derivatives or antibody fragments comprise about 23% to about 46%, about 23% to about 39% or about 31% to about 46% of total antibodies, antibody derivatives or antibody fragments. For example, the percentage of Fc-containing proteins, such as antibodies, with non-glycosylated heavy chains comprise about 5 to about 7%, and other ranges are provided herein. The cells can be cultured for about 10-15 days, preferably about 14 days. The pCO₂ conditions can be between about 30 mmHg and about 210 mmHg, 50 mmHg to 200 mmHg, 60 mmHg to 190 mmHg, 70 mmHg to 180 mmHg, 80 mmHg to 170 mmHg, 90 mmHg to 160 mmHg, 100 mmHg to 150 mmHg, 110 mmHg to 140 mmHg, 120 mmHg to 140 mmHg, 120 mmHg to 130 mmHg or any value within these ranges during the culturing, which is preferably maintained by CO₂ sparging, and can be measured using a CO₂ electrode. As determined by the skilled person in view of the teachings contained herein, pCO₂ can by changed during the culturing process by varying CO₂ sparging, air or other sparging, and/or bioreactor pressure.

The cells can be any suitable mammalian cell, including CHO, BHK, HEK293, HeLa, Human Amniotic, Per.C6 and Sp2/0 cells. Fc-containing proteins, such as antibodies, antibody derivatives and antibody fragments produced thereby are inventions as provided herein.

The Fc-containing proteins can be antibodies, such as antibodies capable of binding PD-1 factor or IL-4 receptors. Preferably, the antibodies are human monoclonal antibodies, preferably all IgG antibodies, including subclasses such as IgG1 and IgG4.

Typically, Fc-containing proteins, such as antibodies, produced according to the inventive teachings contained herein will have acidic charge variants constituting 20%-50% of total Fc-containing proteins, more particularly 20%-47%, 23%-45%, 25%-40%, 28%-37%, 28%-35%, 29%-34%, 30%-33% or any whole or fractional value within these ranges. The Fc-containing proteins will have main peak forms constituting 38%-70% of total Fc-containing proteins, more particularly 45%-70%, 50%-65%, 55%-60% or any whole or fractional value within these ranges. The Fc-containing proteins will have basic charge variants constituting 1%-40% of total Fc-containing proteins, more particularly 2%-35%, 3%-30%, 4%-25%, 5%-20%, 6%-15%, 7%-12%, 7.5%-10%, 8%-9% or any whole or fractional value within these ranges.

Acidic charge variant fractions of the overall products can be controlled, preferably lessened, according to the inventions by ranges of 0.1% to 10% or any whole or fractional value within these ranges. See, for example, Table 1. More particularly, the acidic variants fractions can be lowered 0.2% to 9%, 0.3% to 8%, 0.4% to 7%, 0.5% to 6%, 0.6% to 5%, 0.7% to 4.75%, 0.75% to 4.5%, 0.8% to 4.25%. 0.9% to 4%, 1% to 3.75%. 1% to 3.5%, 1% to 3.25%, 1% to 3%, 1% to 2.75%, 1.25% to 2.5%, 1.25% to 2.25%, 1.25% to 2%, 1.5% to 2%, 1.5% to 1.75% or any whole or fractional value within these ranges. Additionally, other ranges include 0.1% to 4%, 0.25% to 4%, 0.25% to 3.75%, 0.25% to 3.5%, 0.25% to 3%, 0.25% to 2.75%, 0.25% to 2.5%, 0.25% to 2.25%, 0.25% to 2%, 0.25% to 1.75%, 0.25% to 1.5%, 0.25% to 1.25%, 0.25% to 1%, 0.25% to 0.75%. 0.25% to 0.5%, 0.5% to 4%, 0.5% to 3.75%, 0.5% to 3.5%, 0.5% to 3%, 0.5% to 2.75%, 0.5% to 2.5%, 0.5% to 2.25%, 0.5% to 2%, 0.5% to 1.75%, 0.5% to 1.5%, 0.5% to 1.25%, 0.5% to 1%, 0.5% to 0.75%, 0.75% to 4%, 0.75% to 3.75%, 0.75% to 3.5%, 0.75% to 3%, 0.75% to 2.75%, 0.75% to 2.5%, 0.75% to 2.25%, 0.75% to 2%, 0.75% to 1.75%, 0.75% to 1.5%, 0.75% to 1.25%, 0.75% to 1%, 1% to 4%, 1% to 3.75%, 1% to 3.5%, 1% to 3%, 1% to 2.75%, 1% to 2.5%, 1% to 2.25%, 1% to 2%, 1% to 1.75%, 1% to 1.5%, 1% to 1.25%, 1.25% to 4%, 1.25% to 3.75%, 1.25% to 3.5%, 1.25% to 3%, 1.25% to 2.75%, 1.25% to 2.5%, 1.25% to 2.25%, 1.25% to 2%, 1.25% to 1.75%, 1.25% to 1.5% or any whole or fractional value within these ranges. For example. acidic charge variants fractions can be changed, preferably lowered, at least 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5% or more, such as up to 5%, 6%, 7%, 8%, 9%, 10%, 11%, 12%, 13%, 14%, 15% or greater.

Basic charge variant fractions of the overall products can be controlled, according to the inventions by ranges of 0.1% to 15% or any whole or fractional value within these ranges. More particularly, the basic charge variants fractions can be altered 0.1% to 14%, 0.1% to 13%, 0.1% to 12%, 0.1% to 11%, 0.2% to 10%, 0.2% to 9%, 0.3% to 8%, 0.4% to 7%, 0.5% to 6%, 0.6% to 5%, 0.7% to 4.75%, 0.75% to 4.5%, 0.8% to 4.25%. 0.9% to 4%, 1% to 3.75%. 1% to 3.5%, 1% to 3.25%, 1% to 3%, 1% to 2.75%, 1% to 2.75%, 1.25% to 2.5%, 1.25% to 2.25%, 1.25% to 2%, 1.5% to 2%, 1.5% to 1.75% or any whole or fractional value within these ranges. Additionally, other ranges include 0.1% to 4%, 0.25% to 4%, 0.25% to 3.75%, 0.25% to 3.5%, 0.25% to 3%, 0.25% to 2.75%, 0.25% to 2.5%, 0.25% to 2.25%, 0.25% to 2%, 0.25% to 1.75%, 0.25% to 1.5%, 0.25% to 1.25%, 0.25% to 1%, 0.25% to 0.75%. 0.25% to 0.5%, 0.5% to 4%, 0.5% to 3.75%, 0.5% to 3.5%, 0.5% to 3%, 0.5% to 2.75%, 0.5% to 2.5%, 0.5% to 2.25%, 0.5% to 2%, 0.5% to 1.75%, 0.5% to 1.5%, 0.5% to 1.25%, 0.5% to 1%, 0.5% to 0.75%, 0.75% to 4%, 0.75% to 3.75%, 0.75% to 3.5%, 0.75% to 3%, 0.75% to 2.75%, 0.75% to 2.5%, 0.75% to 2.25%, 0.75% to 2%, 0.75% to 1.75%, 0.75% to 1.5%, 0.75% to 1.25%, 0.75% to 1%, 1% to 4%, 1% to 3.75%, 1% to 3.5%, 1% to 3%, 1% to 2.75%, 1% to 2.5%, 1% to 2.25%, 1% to 2%, 1% to 1.75%, 1% to 1.5%, 1% to 1.25%, 1.25% to 4%, 1.25% to 3.75%, 1.25% to 3.5%, 1.25% to 3%, 1.25% to 2.75%, 1.25% to 2.5%, 1.25% to 2.25%, 1.25% to 2%, 1.25% to 1.75%, 1.25% to 1.5% or any whole or fractional value within these ranges. Basic charge variants fractions can be altered at least 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5% or more, such as up to 5%, 6%, 7%, 8%, 9%, 10%, 11%, 12%, 13%, 14%, 15% or greater.

Fc-containing proteins, such as antibodies, produced according to the inventive teachings contained herein typically will have the percentage of non-glycosylated heavy chains (NGHC) present in 3%-8% of total Fc-containing proteins, more particularly 4%-7%, 5%-7% and 5%-6.5%, 5%-6%, 5%-5.75%, 5%-5.5% or any whole or fractional value within these ranges.

Fc-containing proteins, such as antibodies, and derivative and fragments of Fc-containing proteins produced by the inventive methods also are part of the inventions provided herein. Antibodies include, but are not limited to, antibodies that are capable of binding to PD-1 factor and antibodies that are capable of binding to the Interleukin 4 receptor.

### BRIEF DESCRIPTION OF THE FIGURES

FIG. 1 depicts pCO₂ levels of Examples 1 and 3. Medium pCO₂ was selected as a mid-point control.

FIG. 2 depicts pCO₂ levels of Examples 2 and 4. Medium pCO₂ was selected as a mid-point control.

FIG. 3 depicts predicted pH levels during production days of the 2 liter bioreactors at air sparing and pH conditions set forth in Table 6. Medium pCO₂ was selected as a mid-point control.

FIG. 4 depicts Region 1(%) actual (y-axis) and Region 1(%) predicted (x-axis). Region 1 is for acidic charge variants.

FIG. 5 sets for the Summary of Fit, Analysis of Variance and Parameter Estimate of the data of FIG. 4.

FIG. 6 depicts Region 2(%) actual (y-axis) and Region 2(%) predicted (x-axis). Region 2 is for main peak forms.

FIG. 7 sets for the Summary of Fit, Analysis of Variance and Parameter Estimate of the data of FIG. 6.

FIG. 8 depicts Region 3(%) actual (y-axis) and Region 3(%) predicted (x-axis). Region 3 is for basic charge variants.

FIG. 9 sets for the Summary of Fit, Analysis of Variance and Parameter Estimate of the data of FIG. 8.

FIG. 10 depicts NGHC actual (y-axis) and NGHC predicted (x-axis).

FIG. 11 sets for the Summary of Fit, Analysis of Variance and Parameter Estimate of the data of FIG. 10.

FIG. 12 depicts viable cell density values over process time (days). The y-axis has values up to 350×10⁵ cells/ml. Medium pCO₂ was selected as a mid-point control.

FIG. 13 depicts cell viability percentage over process time (days). Medium pCO₂ was selected as a mid-point control.

FIG. 14 depicts pH values over process time (days). Medium pCO₂ was selected as a mid-point control.

FIG. 15 depicts pCO₂ values over process time (days). Medium pCO₂ was selected as a mid-point control.

FIG. 16 depicts glucose values over process time (days). Medium pCO₂ was selected as a mid-point control.

FIG. 17 depicts potassium values over process time (days). Medium pCO₂ was selected as a mid-point control.

FIG. 18 depicts sodium values over process time (days). Medium pCO₂ was selected as a mid-point control.

FIG. 19 depicts osmolality values over process time (days). Medium pCO₂ was selected as a mid-point control.

FIG. 20 depicts glutamate values over process time (days). Medium pCO₂ was selected as a mid-point control.

FIG. 21 depicts lactate values over process time (days). Medium pCO₂ was selected as a mid-point control.

FIG. 22 depicts ammonia values over process time (days). Medium pCO₂ was selected as a mid-point control.

FIG. 23 depicts glutamine values over process time (days). Medium pCO₂ was selected as a mid-point control.

FIG. 24 depicts pCO₂ in mmHg (y-axis) over process time (days) from Example 6. Medium pCO₂ was selected as a mid-point control. TEMP refers to physiologic temperature for the cells as described herein.

### DETAILED DESCRIPTION OF THE INVENTIONS

Unless defined otherwise, all technical and scientific terms used herein have the same meaning as commonly understood by one of ordinary skill in the art to which these inventions belong.

### Definitions

The term “about” in the context of numerical values and ranges refers to values or ranges that approximate or are close to the recited values or ranges such that the invention can perform, such as having a sought rate, amount, density, degree, increase, decrease, percentage, value or presence of a form, variant, temperature or amount of time, as is apparent from the teachings contained herein. Thus, this term encompasses values beyond those simply resulting from systematic error. For example, “about” can signify values either above or below the stated value in a range of approx. +/−10% or more or less depending on the ability to perform.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

Antibodies are often used as therapeutic biomolecules. An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDRI, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

“Acidic charge variants” are Fc-containing protein (for example, antibody) variants that have a lower p/than the main peak form of the Fc-containing protein. Acidic charge variants tend to have more negative charges.

“Basic charge variants” are Fc-containing protein (for example, antibody) variants that have a higher p/than the main peak form of the Fc-containing protein. Basic charge variants tend to have more positive charges or less negative charges.

“Main peak forms” of Fc-containing proteins (for example, antibodies) are the predominant forms of the Fc-containing protein and have a p/between the acidic charge variants and the basic charge variants.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, and vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1- FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FRI, CDRI, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type organism) appears between two framework regions in a variable region of a light or a heavy chain of an immunoglobulin molecule (for example, an antibody or a T cell receptor). A CDR can be encoded by, for example, a germline sequence or a rearranged or unrearranged sequence, and, for example, by a naive or a mature B cell or a T cell. In some circumstances (for example, for a CDR3), CDRs can be encoded by two or more sequences (for example, germline sequences) that are not contiguous (for example, in a nucleic acid sequence that has not been rearranged) but are contiguous in a B cell nucleic acid sequence, for example, as the result of splicing or connecting the sequences (for example, V-D-J recombination to form a heavy chain CDR3).

“Antibody derivatives and fragments” include, but are not limited to: antibody fragments (for example, ScFv-Fc, dAB-Fc, half antibodies), multispecifics (for example, IgG-ScFv, IgG-dab, ScFV-Fc-ScFV, tri-specific).

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcγR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

Fc-containing proteins, such as antibodies, can comprise modifications in immunoglobulin domains, including where the modifications affect one or more effector function of the binding protein (for example, modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein (for example, an antibody) and exhibits enhanced serum half-life (as compared with the same Fc-containing protein without the recited modification(s)) and have a modification at position 250 (for example, E or Q); 250 and 428 (for example, L or F); 252 (for example, L/Y/F/W or T), 254 (for example, S or T), and 256 (for example, S/R/Q/E/D or T); or a modification at 428 and/or 433 (for example, L/R/SI/P/Q or K) and/or 434 (for example, H/F or Y); or a modification at 250 and/or 428; or a modification at 307 or 308 (for example, 308F, V308F), and 434. In another example, the modification can comprise a 428L (for example, M428L) and 434S (for example, N434S) modification; a 428L, 2591 (for example, V259I), and a 308F (for example, V308F) modification; a 433K (for example, H433K) and a 434 (for example, 434Y) modification; a 252, 254, and 256 (for example, 252Y, 254T, and 256E) modification; a 250Q and 428L modification (for example, T250Q and M428L); a 307 and/or 308 modification (for example, 308F or 308P).

“Culture mediums (media)” are aqueous and include minerals, buffer salts, nutrients and other additives needed to support to growth of cells and the production of proteins in culture, such as in bioreactors.

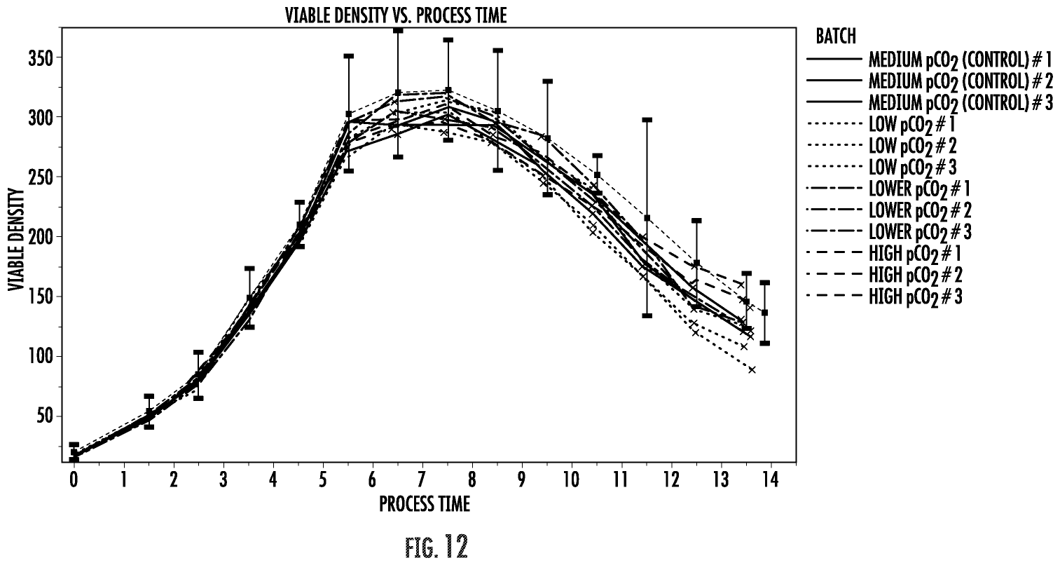

“Peak viable cell density” or “peak VCD” refers to the peak density of the cells during culturing. See FIG. 12.

“Sparging” refers to pumping a gas into a culture medium. The gas can be CO₂, air or other gas. CO₂ sparging will increase pCO₂. Air sparging and nitrogen sparging will decrease pCO₂. Sparging rates are determined based upon the size of the bioreactor, and the rates are typically measured in cubic centimeters per minute (ccm) in small bioreactors. In large bioreactors utilized for commercial production (typically 1,000 to 10,000 liters), sparging rates are measured in standard liters per minute (slpm).

“Protein products” refers to the proteins of interest, such as an Fc-containing proteins (for example, antibodies). Protein products can be produced by cells in culture, usually engineered mammalian cells. Typically, the cells in culture, such as in a bioreactor, will produce proteins of interest, and those proteins will become the protein product. The protein product can be subject to later purification, characterization, sterilization, formulation and other finishing steps, such as concentration or lyophilization, and ultimately packaging to form a finished protein product. Proteins products include formulation drug substances (FDS).

All numerical limits and ranges set forth herein include all numbers or values thereabout or there between of the numbers of the range or limit. The ranges and limits described herein expressly denominate and set forth all integers, decimals and fractional values defined and encompassed by the range or limit.

### DETAILED DESCRIPTION

Antibody charge variants include acidic variants and basic variants. Charge variants can be caused by enzymatic modifications, including deamidation and sialylation that increase net negative charge on the antibodies, which decreases p/values and form acidic variants. Additionally, lysine cleavage from the C-terminus causes loss of net positive charge and leads to formation of acidic variants. Acidic variants also can occur via creation of covalent moieties like glycation, where glucose or lactose react with the primary amine of a lysine residue. Formation of the basic variants are caused by the presence of C-terminal lysine or glycine amidation, succinimide formation, amino acid oxidation or removal of sialic acid. These provide for the addition of positive charges or elimination of negative charges, and thereby increase p/values. See Khawli et al., mAbs 2:6, 613-624 (2010).

The present inventions provide approaches for controlling the population of charge variants (acidic and basic) of proteins and glycosylation variants produced in mammalian cell culture. Embodiments include production of Fc-containing proteins, which include antibodies and fragments and derivatives thereof. The inventions allow for this control by selecting carbon dioxide concentration (pCO₂) of the media during production. NGHC also can be controlled, but via pH.

Aside from pCO₂ levels as taught herein, standard conditions and media can be employed. Typically, cells will be cultured under physiologic conditions, such as temperatures around 36° C. to 38° C., preferably 36° C. to 37° C.

Typically, Fc-containing proteins (for example, antibodies) produced according to the inventive teachings contained herein will have acidic charge variants constituting 20%-50% of total Fc-containing proteins, more particularly 20%-47%, 23%-45%, 25%-40%, 28%-37%, 28%-35%, 29%-34%, 30%-33% or any whole or fractional value within these ranges. The Fc-containing proteins will have main peak forms constituting 38%-70% of total Fc-containing proteins, more particularly 45%-70%, 50%-65%, 55%-60% or any whole or fractional value within these ranges. The Fc-containing proteins will have basic charge variants constituting 1%-40% of total Fc-containing proteins, more particularly 2%-35%, 3%-30%, 4%-25%, 5%-20%, 6%-15%, 7%-12%, 7.5%-10%, 8%-10%, 8%-9% or any whole or fractional value within these ranges.

Fc-containing proteins (for example, antibodies) produced according to the inventive teachings contained herein typically will have the percentage of non-glycosylated heavy chains (NGHC) present in 3%-8% of total Fc-containing proteins, more particularly 4%-7%, 5%-7% and 5%-6.5%, 5%-6%, 5%-5.75%, 5%-5.5% or any whole or fractional value within these ranges.

Acidic charge variant fractions of the overall products can be controlled, preferably lessened, according to the inventions by ranges of 0.1% to 10% or any whole or fractional value within these ranges. See, for example, Table 1. More particularly, the acidic variants fractions can be lowered 0.2% to 9%, 0.3% to 8%, 0.4% to 7%, 0.5% to 6%, 0.6% to 5%, 0.7% to 4.75%, 0.75% to 4.5%, 0.8% to 4.25%. 0.9% to 4%, 1% to 3.75%. 1% to 3.5%, 1% to 3.25%, 1% to 3%, 1% to 2.75%, 1% to 2.75%, 1.25% to 2.5%, 1.25% to 2.25%, 1.25% to 2%, 1.5% to 2%, 1.5% to 1.75% or any whole or fractional value within these ranges. Additionally, other ranges include 0.1% to 4%, 0.25% to 4%, 0.25% to 3.75%, 0.25% to 3.5%, 0.25% to 3%, 0.25% to 2.75%, 0.25% to 2.5%, 0.25% to 2.25%, 0.25% to 2%, 0.25% to 1.75%, 0.25% to 1.5%, 0.25% to 1.25%, 0.25% to 1%, 0.25% to 0.75%. 0.25% to 0.5%, 0.5% to 4%, 0.5% to 3.75%, 0.5% to 3.5%, 0.5% to 3%, 0.5% to 2.75%, 0.5% to 2.5%, 0.5% to 2.25%, 0.5% to 2%, 0.5% to 1.75%, 0.5% to 1.5%, 0.5% to 1.25%, 0.5% to 1%, 0.5% to 0.75%, 0.75% to 4%, 0.75% to 3.75%, 0.75% to 3.5%, 0.75% to 3%, 0.75% to 2.75%, 0.75% to 2.5%, 0.75% to 2.25%, 0.75% to 2%, 0.75% to 1.75%, 0.75% to 1.5%, 0.75% to 1.25%, 0.75% to 1%, 1% to 4%, 1% to 3.75%, 1% to 3.5%, 1% to 3%, 1% to 2.75%, 1% to 2.5%, 1% to 2.25%, 1% to 2%, 1% to 1.75%, 1% to 1.5%, 1% to 1.25%, 1.25% to 4%, 1.25% to 3.75%, 1.25% to 3.5%, 1.25% to 3%, 1.25% to 2.75%, 1.25% to 2.5%, 1.25% to 2.25%, 1.25% to 2%, 1.25% to 1.75%, 1.25% to 1.5% or any whole or fractional value within these ranges. For example. acidic charge variants fractions can be changed, preferably lowered, at least 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5% or more, such as up to 5%, 6%, 7%, 8%, 9%, 10%, 11%, 12%, 13%, 14%, 15% or greater.

Basic charge variant fractions of the overall products can be controlled according to the inventions by ranges of 0.1% to 10% or any whole or fractional value within these ranges. More particularly, the basic charge variants fractions can be altered 0.2% to 9%, 0.3% to 8%, 0.4% to 7%, 0.5% to 6%, 0.6% to 5%, 0.7% to 4.75%, 0.75% to 4.5%, 0.8% to 4.25%. 0.9% to 4%, 1% to 3.75%. 1% to 3.5%, 1% to 3.25%, 1% to 3%, 1% to 2.75%, 1% to 2.75%, 1.25% to 2.5%, 1.25% to 2.25%, 1.25% to 2%, 1.5% to 2%, 1.5% to 1.75% or any whole or fractional value within these ranges. Additionally, other ranges include 0.1% to 4%, 0.25% to 4%, 0.25% to 3.75%, 0.25% to 3.5%, 0.25% to 3%, 0.25% to 2.75%, 0.25% to 2.5%, 0.25% to 2.25%, 0.25% to 2%, 0.25% to 1.75%, 0.25% to 1.5%, 0.25% to 1.25%, 0.25% to 1%, 0.25% to 0.75%. 0.25% to 0.5%, 0.5% to 4%, 0.5% to 3.75%, 0.5% to 3.5%, 0.5% to 3%, 0.5% to 2.75%, 0.5% to 2.5%, 0.5% to 2.25%, 0.5% to 2%, 0.5% to 1.75%, 0.5% to 1.5%, 0.5% to 1.25%, 0.5% to 1%, 0.5% to 0.75%, 0.75% to 4%, 0.75% to 3.75%, 0.75% to 3.5%, 0.75% to 3%, 0.75% to 2.75%, 0.75% to 2.5%, 0.75% to 2.25%, 0.75% to 2%, 0.75% to 1.75%, 0.75% to 1.5%, 0.75% to 1.25%, 0.75% to 1%, 1% to 4%, 1% to 3.75%, 1% to 3.5%, 1% to 3%, 1% to 2.75%, 1% to 2.5%, 1% to 2.25%, 1% to 2%, 1% to 1.75%, 1% to 1.5%, 1% to 1.25%, 1.25% to 4%, 1.25% to 3.75%, 1.25% to 3.5%, 1.25% to 3%, 1.25% to 2.75%, 1.25% to 2.5%, 1.25% to 2.25%, 1.25% to 2%, 1.25% to 1.75%, 1.25% to 1.5% or any whole or fractional value within these ranges. Basic charge variants fractions can be altered at least 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5% or more, such as up to 5%, 6%, 7%, 8%, 9%, 10%, 11%, 12%, 13%, 14%, 15% or greater.

Typically, CO₂ concentrations during fermentation come from two sources, namely atmospheric CO₂ and CO₂ produced by the cells via respiration. The inventions advantageously can employ additional CO₂ to control charge variants. Although not bound by any theory, it is believed that increasing CO₂ levels in media leads to increase in intracellular CO₂, which is solely or jointly responsible for charge variation. This effect is separate from any decrease in pH possibly due to the formation of carbonic acid or other acidic chemicals.

Carbon dioxide concentration can be increased using CO₂ sparging or by lowering air sparging. CO₂ sparging increases pCO₂. Should a decrease in carbon dioxide concentration be desired, sparging can be undertaken with other gasses, including air. Air sparging and nitrogen sparging decreases pCO₂. Reducing pressure in production bioreactors results in reduced solubility of oxygen; this in turn requires greater sparging of oxygen to maintain a dissolved oxygen (DO) set point and increased gas flow rate, which drives off pCO₂ from the culture medium.

Carbon dioxide concentration can be measured using a CO₂ electrode, also referred to as a Severinghaus electrode. More advanced systems are commercially available, such as the BioProfile® FLEX and FLEX 2 Analyzers. Charge variants can be measured using Imaged Capillary Isoelectric Focusing (iCIEF) and ion exchange chromatography with elution by salt gradient. NGHC can be measured by reduced capillary electrophoresis (CE)-SDS.

The present inventions are amenable for use with mammalian cell culture. Exemplary cell lines are CHO, Per.C6 cells, Sp2/0 cells, and HEK293 cells. CHO cells include, but are not limited to, CHO-ori, CHO-K1, CHO-s, CHO-DHB11, CHO-DXB11, CHO-K1SV, and mutants and variants thereof. HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof. Other suitable cells include, but are not limited to BHK (baby hamster kidney) cells, HeLa cells and Human Amniotic cells, such as Human Amniotic Epithelial cells.

The inventions can be employed in the production of biological and pharmaceutical products, including next-generation versions of existing biological and pharmaceutical products produced in cell culture. A wide range of protein-based therapeutics, such as monoclonal antibody-based therapeutics, can be produced according to the inventions. For example, cells comprising requisite DNA sequences encoding antibodies, including but not limited to the antibodies identified below, can be grown in culture according the present inventions.

The following identifies and describes proteins made in cell culture that can be produced according to the present inventions. Cells comprising the requisite DNA encoding these proteins can be cultured for production according to the present inventions.

For example, for antibody production, the inventions are amendable for research and production use for diagnostics and therapeutics based upon all major antibody classes, namely IgG, IgA, IgM, IgD and IgE. IgG is a preferred class, and includes subclasses IgG1 (including IgG1λ and IgG1κ), IgG2, IgG3, and IgG4. Further antibody embodiments include a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody. Derivatives, components, domains, chains and fragments of the above also are included.

Further antibody embodiments include a human antibody, a humanized antibody, a chimeric antibody, a monoclonal antibody, a multispecific antibody, a bispecific antibody, a trispecific antibody, an antigen binding antibody fragment, a single chain antibody; a diabody, triabody or tetrabody, a Fab fragment or a F(ab)2 fragment, an IgD antibody, an IgE antibody, an IgM antibody, an IgG antibody, an IgG1 antibody. an IgG2 antibody, an IgG3 antibody, or an IgG4 antibody. In one embodiment, the antibody is an IgG1 antibody. In an embodiment, the antibody is an IgG2 antibody. In one embodiment, the antibody is an IgG4 antibody. In another embodiment, the antibody is a chimeric IgG2/IgG4 antibody. In another embodiment, the antibody is a chimeric IgG2/IgG1 antibody. In another embodiment, the antibody is a chimeric IgG2/IgG1/IgG4 antibody.

In additional embodiments, the antibody is selected from the group consisting of an anti-Programmed Cell Death 1 antibody (for example an anti-PD1 antibody as described in U.S. Pat. Appln. Pub. No. US2015/0203579A1), an anti-Programmed Cell Death Ligand-1 (for example an anti-PD-L1 antibody as described in in U.S. Pat. Appln. Pub. No. US2015/0203580A1), an anti-DII4 antibody, an anti-Angiopoetin-2 antibody (for example an anti-ANG2 antibody as described in U.S. Pat. No. 9,402,898), an anti-Angiopoetin-Like 3 antibody (for example an anti-AngPtl3 antibody as described in U.S. Pat. No. 9,018,356), an anti-platelet derived growth factor receptor antibody (for example an anti-PDGFR antibody as described in U.S. Pat. No. 9,265,827), an anti-Erb3 antibody, an anti-Prolactin Receptor antibody (for example anti-PRLR antibody as described in U.S. Pat. No. 9,302,015), an anti-Complement 5 antibody (for example an 25 anti-05 antibody as described in U.S. Pat. Appln. Pub. No US2015/0313194A1), an anti-TNF antibody, an anti-epidermal growth factor receptor antibody (for example an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (for example an anti-PCSK9 antibody as described in U.S. Pat. No. 8,062,640 or U.S. Pat. Appln. Pub. No. US2014/0044730A1), an anti-Growth And Differentiation Factor-8 antibody (for example an anti-GDF8 antibody, also known as anti-myostatin antibody, as described in U.S. Pat. No. 8,871,209 or 9,260,515), an anti-Glucagon Receptor (for example anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g an anti-IL4R antibody as described in U.S. Pat. Appln. Pub. No. US2014/0271681A1 or U.S. Pat. No. 8,735,095 or 8,945,559), an anti-interleukin 6 receptor antibody (for example an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example anti-IL33 antibody as described in U.S. Pat. Appln. Pub. Nos. US2014/0271658A1 or US2014/0271642A1), an anti-Respiratory syncytial virus antibody (for example anti-RSV antibody as described in U.S. Pat. Appln. Pub. No. US2014/0271653A1), an anti-Cluster of differentiation 3 (for example an anti-CD3 antibody, as described in U.S. Pat. Appln. Pub. Nos. US2014/0088295A1 and US20150266966A1, and in U.S. Application No. 62/222,605), an anti-Cluster of differentiation 20 (for example an anti-CD20 antibody as described in U.S. Pat. Appln. Pub. Nos.

US2014/0088295A1 and US20150266966A1, and in U.S. Pat. No. 7,879,984), an anti-CD19 antibody, an anti-0028 antibody, an anti-Cluster of Differentiation 48 (for example anti-0048 antibody as described in U.S. Pat. No. 9,228,014), an anti-Fel d1 antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S. Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. Appln. Pub. No. US2016/0017029 and U.S. Pat. Nos. 8,309,088 and 9,353,176) and an anti-Activin A antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (for example, an anti-CD3×anti-Muc16 bispecific antibody), and an anti-CD3×anti-Prostate-specific membrane antigen bispecific antibody (for example, an anti-CD3×anti-PSMA bispecific antibody). See also U.S. Patent Publication No. US 2019/0285580 A1. Also included are a Met×Met antibody, an agonist antibody to NPR1, an LEPR agonist antibody, a BCMA×CD3 antibody, a MUC16×CD28 antibody, a GITR antibody, an IL-2Rg antibody, an EGFR×CD28 antibody, a Factor XI antibody, antibodies against SARS-CoC-2 variants, a Fel d 1 multi-antibody therapy, a Bet v 1 multi-antibody therapy. Derivatives, components, domains, chains and fragments of the above also are included.

Cells that produce exemplary antibodies can be cultured according to the inventions. Exemplary antibodies include Alirocumab, Atoltivimab, Maftivimab, Odesivimab, Odesivivmab-ebgn, Casirivimab, Imdevimab, Cemiplimab and Cemiplimab-rwlc (human IgG4 monoclonal antibody that binds PD-1), Dupilumab (human monoclonal antibody of the IgG4 subclass that binds to the IL-4R alpha (a) subunit and thereby inhibits Interleukin 4 (IL-4) and Interleukin 13 (IL-13) signalling), Evinacumab, Evinacumab-dgnb, Fasinumab, Fianlimab, Garetosmab, Itepekimab Nesvacumab, Odrononextamab, Pozelimab, Sarilumab, Trevogrumab, and Rinucumab.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Phillipines.

A biosimilar in the U.S. is currently described as (A) a biological product is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product that is shown that may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to an reference product already approved in Japan. In India, biosimilars currently are referred to as “similar biologics,” and refer to a similar biologic product is that which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the inventions.

Typically, culturing can occur for about 10-15 days, preferably about 12-14 days. The pCO₂ conditions are between 30 mmHg and 210 mmHg of CO₂, 50 mmHg to 200 mmHg, 60 mmHg to 190 mm Hg, 70 mmHg to 180 mmHg, 80 mmHg to 170 mmHg, 90 mmHg to 160 mmHg, 100 mmHg to 150 mmHg, 110 mmHg to 140 mmHg, 120 mmHg to 140 mmHg, 120 mmHg to 130 mmHg or any value within these ranges during the culturing. The inventions can provide Fc-containing protein products, such as antibodies, wherein the main peak (considered about neutral) form comprises 38% to 65% of total Fc-containing proteins, the acidic variant of the Fc-containing proteins comprises 20% to 47% of total Fc-containing proteins and the basic variant of the Fc-containing proteins comprises up to 36% of total Fc-containing proteins. In the case of antibodies, the inventions can provide products where the main peak form of antibodies produced by the cells comprises between 50% to 70% of total antibodies, the acidic variant of the antibodies comprises 20% to 47% of total antibodies and the basic variant of the antibodies comprises up to 15% of total antibodies.

The inventions are further described by the following examples, which are illustrative of the many aspects of the invention, but do not limit the inventions in any manner.

### Example 1—Culture pCO₂ can be Increased in Order to Decrease Acidic Variants and Increase Main Peak Forms in a Preparation of a Human IgG4 Monoclonal Antibody that Binds to Programmed Cell Death Protein 1 (PD-1) Factor

The culture media was inoculated with CHO cells at a concentration of 18×10⁶ cells/ml and allowed to grow in a fed-batch process. Once the cells reached peak concentration (30×10⁶ cells/ml) on Day 7, the high pCO₂ bioreactors where sparged with additional CO₂ to increase pCO₂ levels above 120 mmHg. The control process implemented a standard production process to maintain pCO₂ levels below 105 mmHg. See FIG. 1. The observed acidic heterogeneity is tabulated below in Table 1, and support high pCO₂ ranges of 31% to 32% for the acidic charge variant and 57% to 60% for the main peak form:

TABLE 1

| Condition | Acidic Variant (%) | Main Peak Form(%) |
|---|---|---|
| Medium pCO₂ | a. 33.9 | a. 55.2 |
| | b. 33.4 | b. 55.1 |
| High pCO₂ | a. 31.8 | a. 57.7 |
| | b. 31.6 | b. 57.8 |
| | c. 31.1 | c. 59.8 |

### Example 2—Culture pCO₂ can be Decreased and Thereby Increase Acidic Variants in a Preparation of a Human IgG4 Monoclonal Antibody that Binds to PD-1 Factor

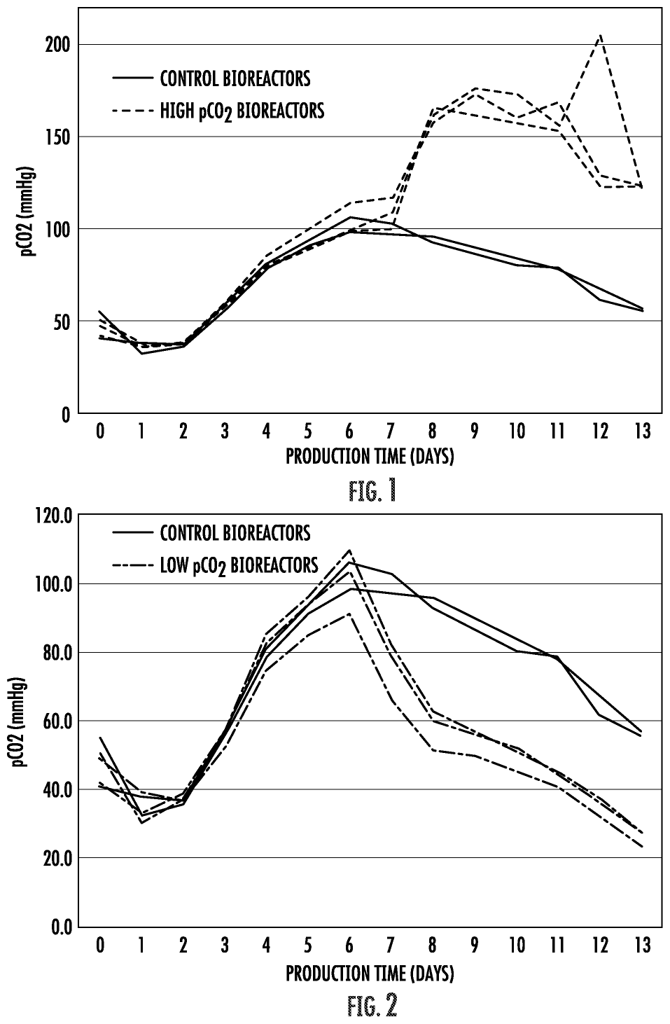

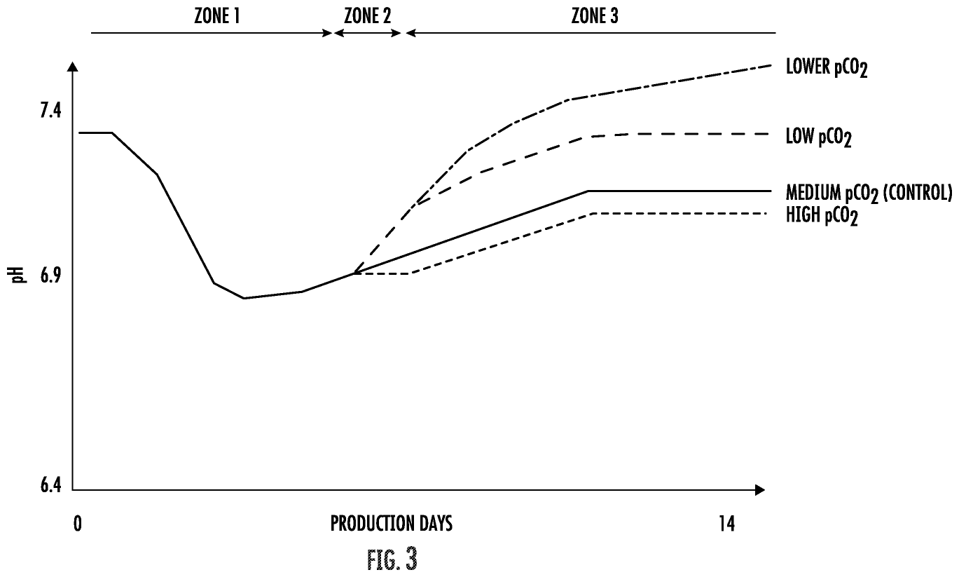

Media was inoculated with CHO cells at a concentration of 18×10⁶ cells/ml and process proceeded in fed batch mode. To drive off and reduce culture pCO₂, on Day 6.5 the air sparge in the replicate bioreactors was increased from 22 ccm to 33 ccm for 24 hours and subsequently increased to 44 ccm from Day 7.5 to harvest. The control replicate bioreactors maintained an air sparge of 22 ccm for the duration of the process. See FIG. 2. The observed acidic variant heterogeneity is tabulated below in Table 2:

TABLE 2

| Condition | Acidic Variant (%) |
|---|---|
| Medium pCO₂ | a. 33.9 |
| | b. 33.4 |
| Low pCO₂ | a. 34.0 |
| | b. 35.2 |
| | c. 34.6 |

This example established that low pCO₂ results in a higher percentage of acidic charge variants.

### Example 3—Increase in Culture pCO₂ has an Association with Increases in the Prevalence of NGHC in a Preparation of a Human IgG4 Monoclonal Antibody that Binds to PD-1 Factor

The culture media was inoculated with CHO cells at a concentration of 18×10⁶ cells/ml and allowed to grow in a fed-batch process. Once the cells reached peak concentration (30×10⁶ cells/ml) on Day 7, the high pCO₂ bioreactors where sparged with additional CO₂ to increase pCO₂ levels above 120 mmHg. The control process implemented a standard production process to maintain pCO₂ levels below 105 mmHg. See FIG. 1. The observed NGHC heterogeneity is tabulated below in Table 3:

TABLE 3

| Condition | NGHC (%) |
|---|---|
| Medium pCO₂ | a. 5.81 |
| | b. 5.77 |
| High pCO₂ | a. 6.64 |
| | b. 6.27 |
| | c. 5.88 |

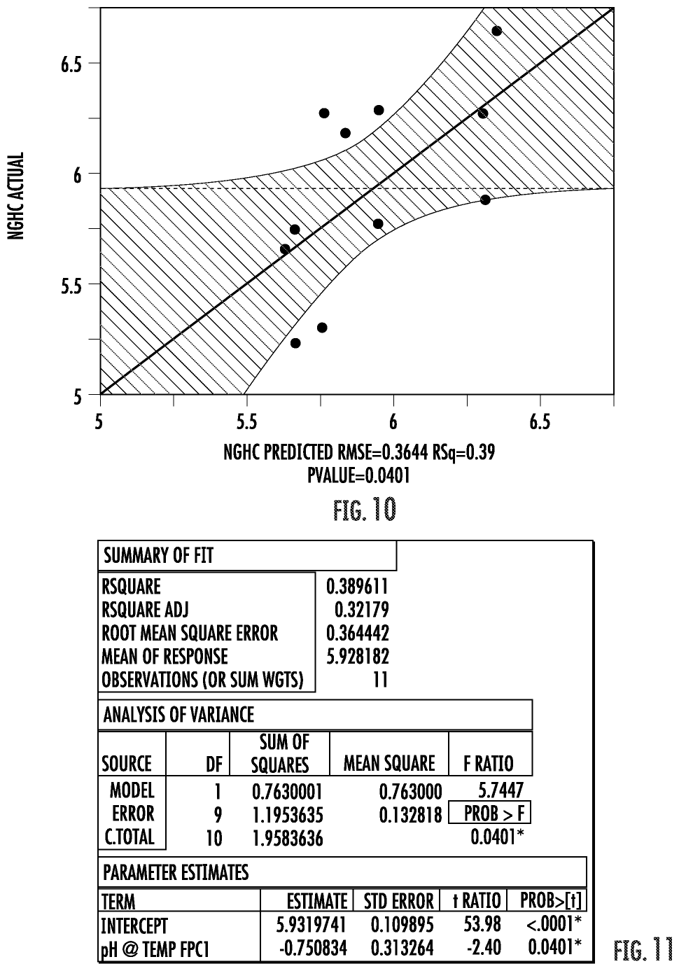

The increase in NGHC was determined to be linked to a decrease in culture pH, and not an effect of pCO₂ per se. See Example 5 and FIGS. 10 and 11.

### Example 4—Decrease in Culture pCO₂ has an Association with Decreases in the Prevalence of NGHC in a Preparation of a Human IgG4 Monoclonal Antibody that can Bind PD-1 Factor

Media was inoculated with CHO cells at a concentration of 18×10⁶ cells/ml and process proceeded in fed batch mode. To drive off and reduce culture pCO₂, on Day 6.5 the air sparge in the replicate bioreactors was increased from 22 ccm to 33 ccm for 24 hours and subsequently increased to 44 ccm from Day 7.5 to harvest. The control replicate bioreactors maintained an air sparge of 22 ccm for the duration of the process. See FIG. 2. The observed NGHC heterogeneity is tabulated below in Table 4:

TABLE 4

| Condition | NGHC (%) |
|---|---|
| Medium pCO₂ | a. 5.81 |
| | b. 5.77 |
| Low pCO₂ | a. 5.75 |
| | b. 5.22 |
| | c. 5.30 |

The decrease in NGHC was determined to be linked to an increase in culture pH, and not an effect of pCO₂ per se. See Example 5 and FIGS. 10 and 11.

### Example 5—Analysis of Culture pCO₂ and pH in a Small Scale Study on the Production of a Human IgG4 Monoclonal Antibody that can Bind PD-1 Factor

The data from a typical large scale production run of an Fc-containing protein (for example, an antibody) using CHO cells is shown below in Table 5.

TABLE 5

| | | FDS | | | | | | | | | | | | | | | |
|---|---|---|---|---|---|---|---|---|---|---|---|---|---|---|---|---|---|
| | | Release | | FDS | | | | | | | | | | | | | |
| | | Acceptance | | Historical | | FDS Lots | | | | | | | | | | | |
| Test | | Criteria | | min-max | | First | | Second | | Third | | Fourth | | Fifth | | Sixth | |
| Charged | | | | | | | | | | | | | | | | | |
| Variant | | | | | | | | | | | | | | | | | |
| Analysis | | | | | | | | | | | | | | | | | |
| a. | % Region 1 | a. | 23-39% | a. | 29-33% | a. | 27 | a. | 28 | a. | 27 | a. | 27 | a. | 28 | a. | 27 |
| b. | % Region 2 | b. | 51-65% | b. | 55-60% | b. | 64 | b. | 64 | b. | 64 | b. | 64 | b. | 66 | b. | 65 |
| c. | % Region 3 | c. | ≤15% | c. | 10-14% | c. | 9 | c. | 9 | c. | 8 | c. | 8 | c. | 7 | c. | 8 |
| Reduced CE-SDS | | | | | | | | | | | | | | | | | |
| a. | % NGHC | a. | N/A | a. | 3.5-6.6% | a. | 7.5 | a. | 7.4 | a. | 7.3 | a. | 6.8 | a. | 7.6 | a. | 8.1 |

A small scale study using 2L fermenters was undertaken to replicate the large scale production of formulation drug substances (FDS). The results from the study described herein are used to demonstrate that alterations in culture pCO₂ and pH, similar to that observed in the 10,000 L production bioreactor, influence the charge variant profile and the occurrence of non-glycosylated heavy chains in a human IgG4 monoclonal antibody that binds PD-1.

The small scale study determined that elevated pCO₂ levels in a production bioreactor caused an observed decrease in iCIEF Region 1 (acidic charge variants) and Region 3 (basic charge variants), and contributed to a concomitant increase in iCIEF Region 2 (main peak form, also known as a main peak variant). The study also concluded that culture pH, not pCO₂ itself, caused the observed change in NGHC profile. These results are discussed with greater specificity below. The study parameters using air sparging and pCO₂ sparging are outlined below in Table 6:

TABLE 6
Air Sparge and CO₂ Sparging Relative to Control Condition

| | Day 0-6.5 | | Day 6.5-7.5 | | Day 7.5-14 | |
|---|---|---|---|---|---|---|
| | | Addi- | | Addi- | | Addi- |
| | | tional | | tional | | tional |
| | | CO₂ | | CO₂ | | CO₂ |
| | Air | Sparg- | Air | Sparg- | Air | Sparg- |
| Condition | Sparge | ing | Sparge | ing | Sparge | ing |
| High | 100% | No | 100% | Yes | 100% | Yes |
| pCO₂ | | | | | | |
| Medium | 100% | No | 100% | No | 100% | No |
| pCO₂ | | | | | | |
| (Control) | | | | | | |
| Low | 100% | No | 118% | No | 136% | No |
| pCO₂ | | | | | | |
| Lower | 100% | No | 150% | No | 200% | No |
| pCO₂ | | | | | | |

The charge variant (iCIEF) and NGHC results for each run are set forth in Table 7. *Note—Medium pCO₂ #3 (viewed as a mid-point control) was removed from further analysis as it represented an outlier that could confound interpretation of the data.

TABLE 7

| | iCIEF Region 1 | iCIEF Region 2 | iCIEF Region 3 | NGHC |
|---|---|---|---|---|
| Condition | (%) | (%) | (%) | (%) |
| Medium pCO₂ #1 | 33.9 | 55.24 | 10.86 | 5.81 |
| Medium pCO₂ #2 | 33.4 | 55.16 | 11.44 | 5.77 |
| Medium pCO₂ #3* | 38.12 | 53.21 | 8.67 | 6.27 |
| High pCO₂ #1 | 31.8 | 57.7 | 10.5 | 6.64 |
| High pCO₂ #2 | 31.69 | 57.86 | 10.44 | 6.27 |
| High pCO₂ #3 | 31.13 | 59.8 | 9.08 | 5.88 |
| Low pCO₂ #1 | 33.13 | 55.12 | 11.75 | 6.27 |
| Low pCO₂ #2 | 30.01 | 55.35 | 11.63 | 5.30 |
| Low pCO₂ #3 | 35.22 | 55.5 | 9.28 | 6.18 |
| Lower pCO₂ #1 | 34.09 | 55.34 | 10.57 | 5.75 |
| Lower pCO₂ #2 | 35.22 | 54.37 | 10.41 | 5.22 |
| Lower pCO₂ #3 | 34.63 | 55.09 | 10.28 | 5.30 |

The cause for charge variants and peak forms, namely iCIEF Region 1 (acidic charge variants), Region 2 (main peak forms) and Region 3 (basic charge variants) is discussed in greater detail below. NGHC also is discussed below.

FIG. 3 shows pH values predicted using the parameters according to Table 6.

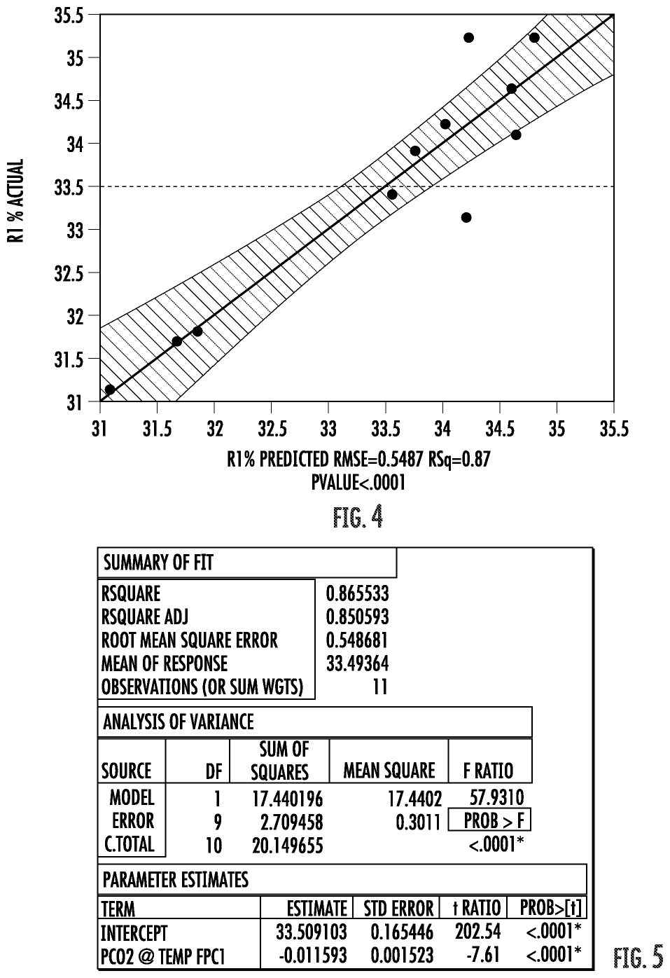

FIG. 4 depicts the data that shows that culture pCO₂ is the only significant term (p<0.0001) in the model for iCIEF Region 1 (R1, acidic charge variants %) and accounts for 87% (R²: 0.87) of the variability in this charge variant such that higher pCO₂ is the sole statistically term associated with lower Region 1(%) (Acidic charge variants). Culture pH was not a statistically significant term of acidic charge variants (Region 1). See FIG. 5.

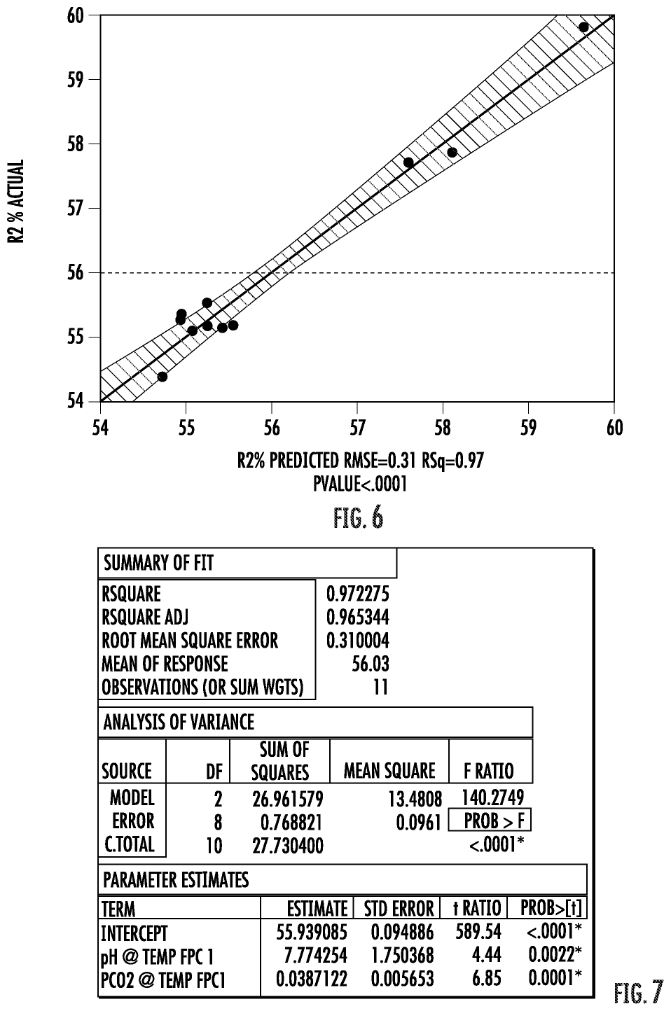

FIG. 6 depicts data that shows that both culture pCO₂ and pH were significant terms (p<0.0001) in the model for iCIEF Region 2 (R2, main peak forms %) and accounts of 97% of the observed variability (R²: 0.97). As such, higher culture pCO₂ and lower culture pH increase the main peak form. See FIG. 7.

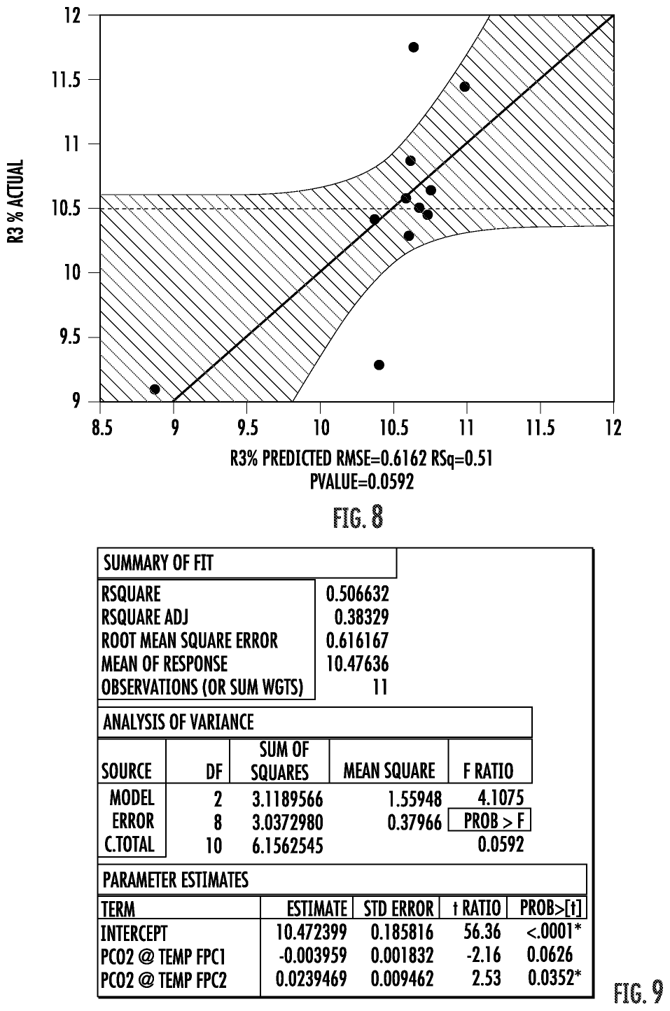

FIG. 8 depicts data that shows that culture pCO₂ was a significant term in the model (p=0.0352) and explained 38% of the variability in Region 3 (R3, basic charge variants %). However, this model was not significant (p=0.0592), possibly due to over-leveraging of a data point. See FIG. 9.

The above data show that charge variants generally are caused in whole or in part by increasing pCO₂ levels. More importantly, increased pCO₂, and not decreased pH, was the only statistically significant term for lowering the percentage of acidic charge variants (Region 1). Thus, for the IgG class, here represented by a human IgG4 monoclonal antibody, increased pCO₂ lowers the percentage of acidic charge variants, and the lowering of the percentage of acidic charge variants is not caused by decreased pH values. See FIGS. 4 and 5.

Finally, FIGS. 10 and 11 depict data that shows that decreased culture pH, and not increased pCO₂ itself, was a significant term in the model (p=0.0401) accounting for 38% of the variability in NGHC(R²: 0.38). Therefore, lower culture pH can influence the NGHC profile. While pCO₂ can have an effect on pH, other media ingredients also have an effect on pH, and thus pH, no matter the cause, is what alters NGHC. Accordingly, less acidic charge variants (%) are due to a phenomenon, pCO₂ itself, that is different from increases in NGHC, which are caused by lowering of pH by any type of acidic molecule.

FIGS. 12 to 23 depict data for:

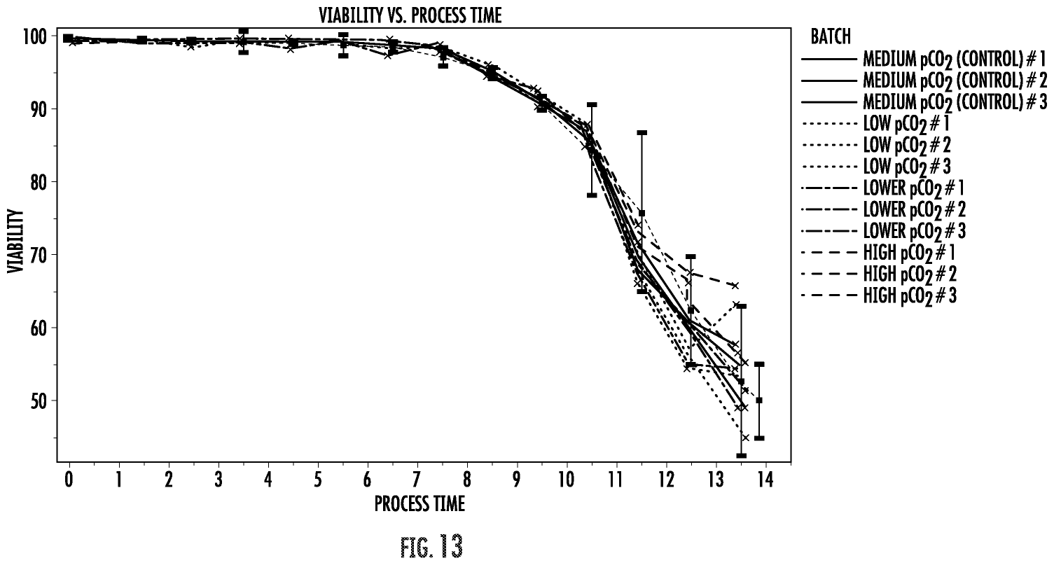

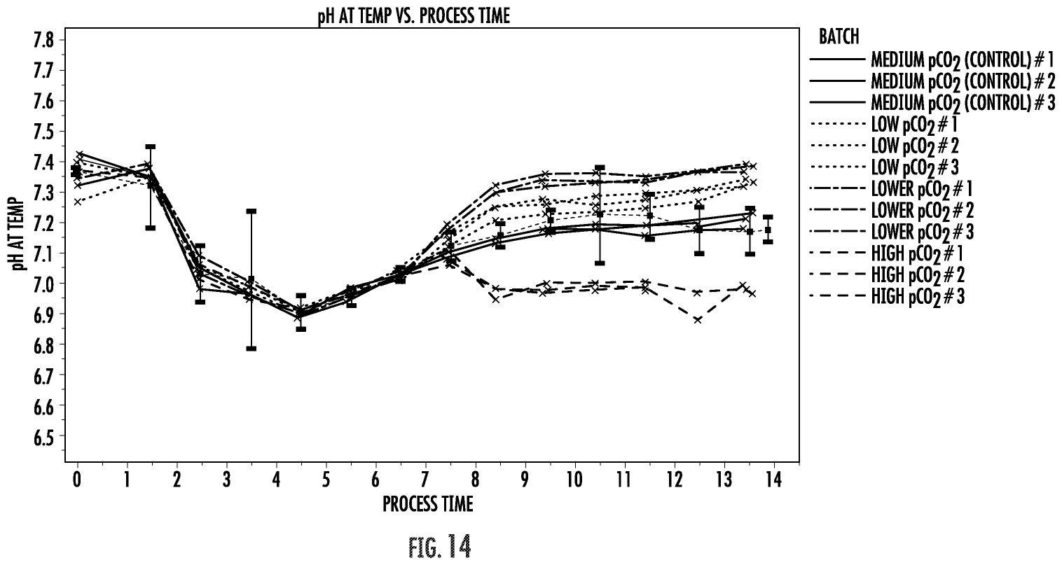

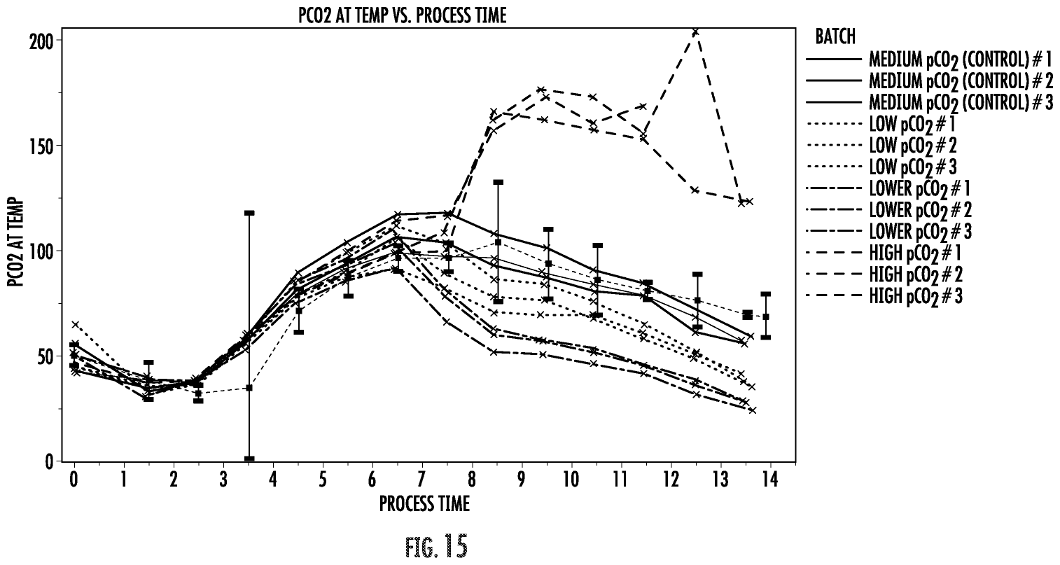

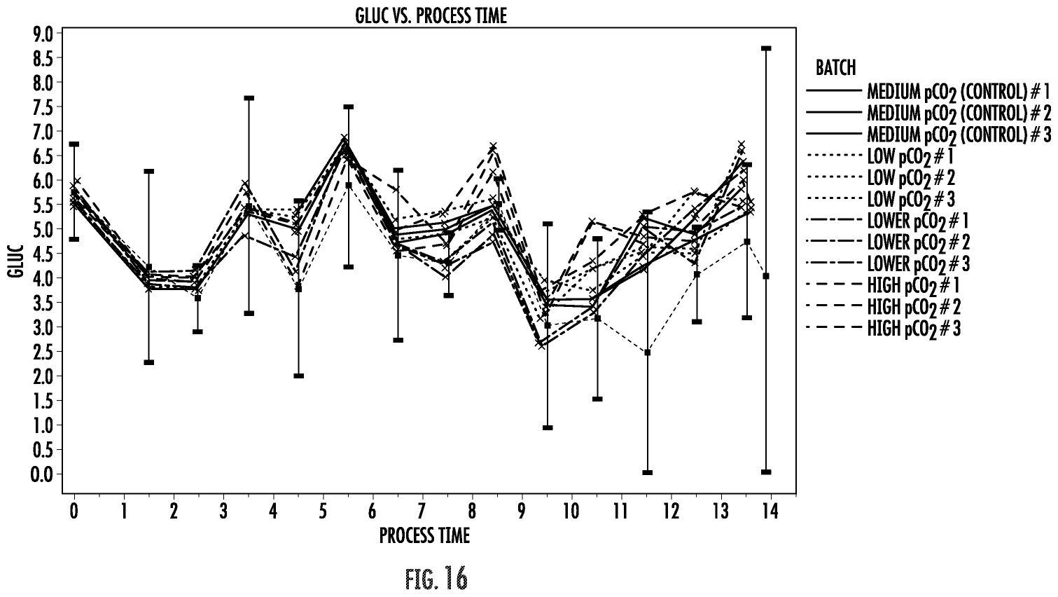

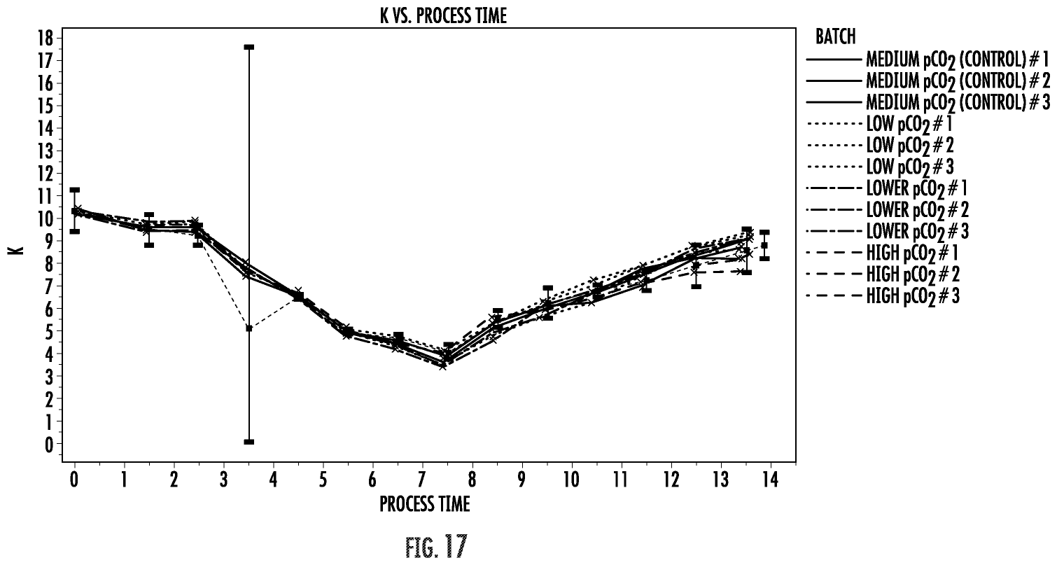

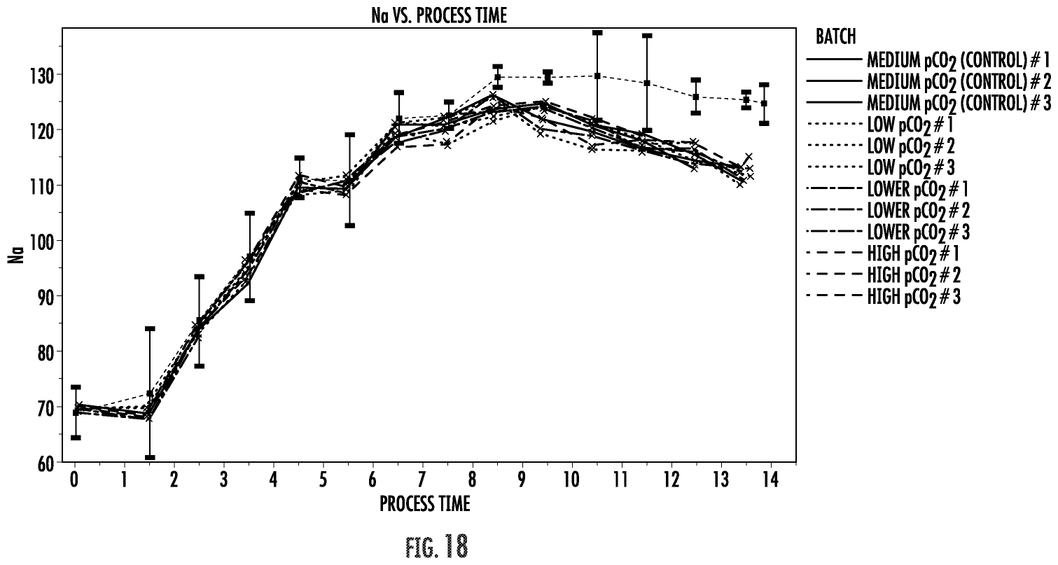

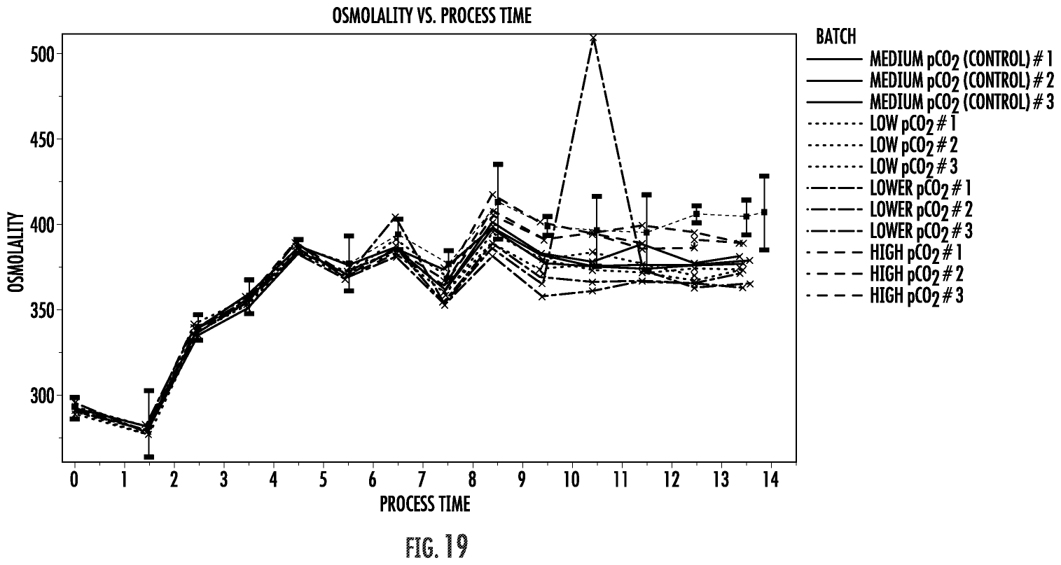

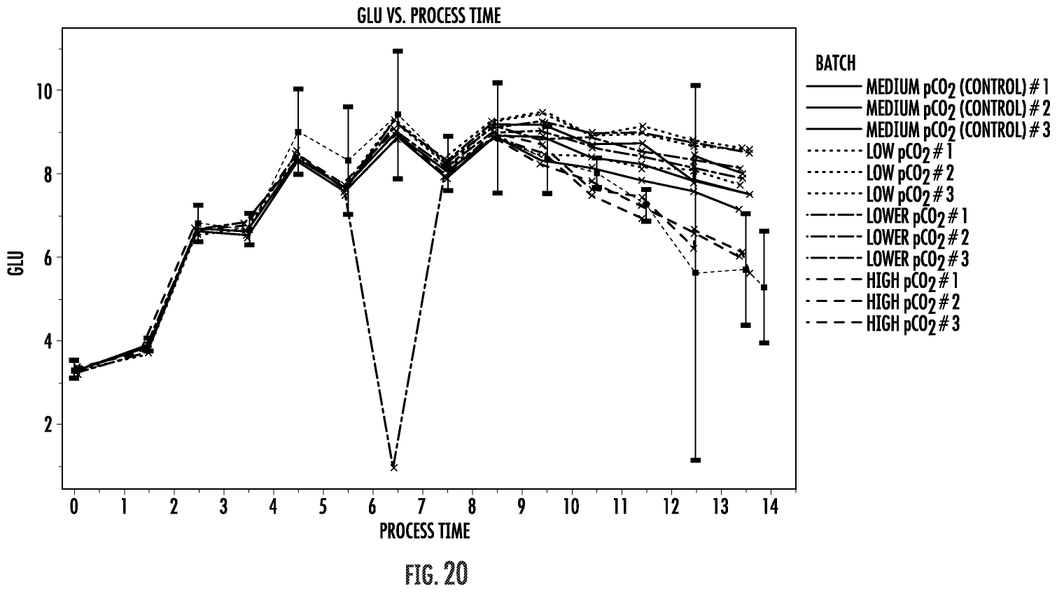

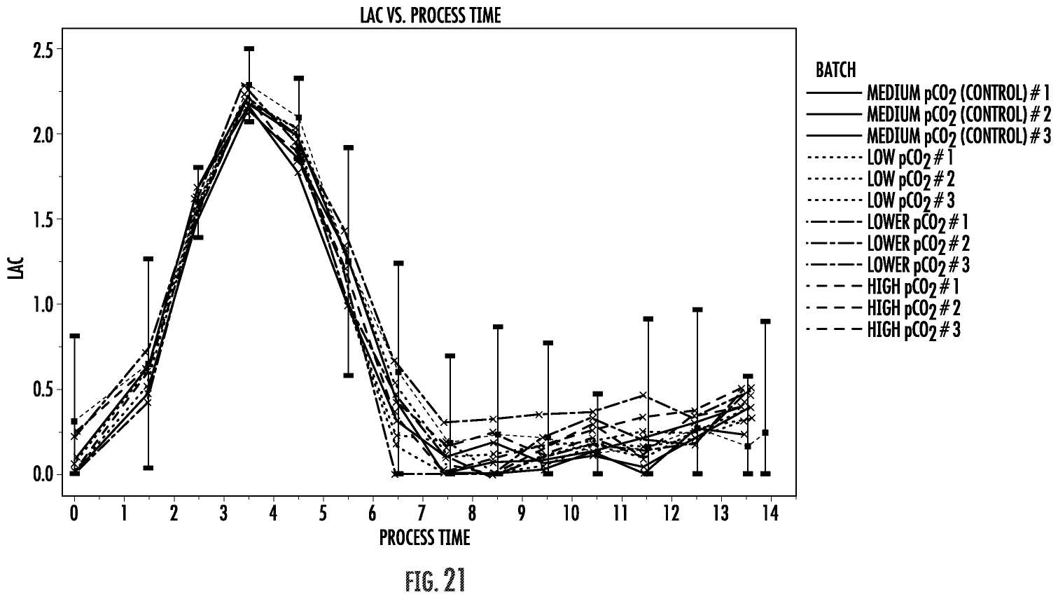

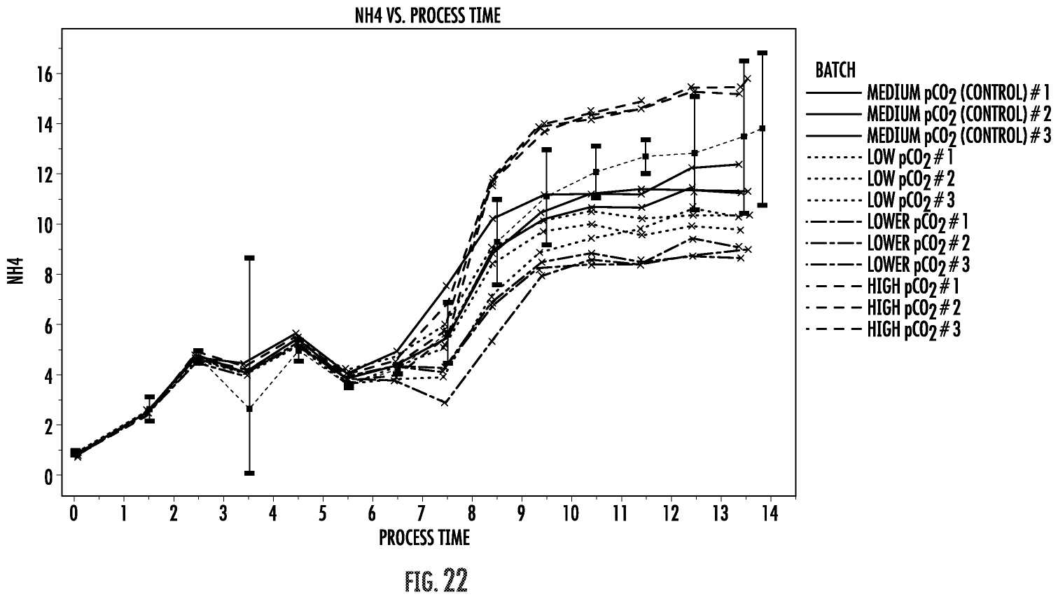

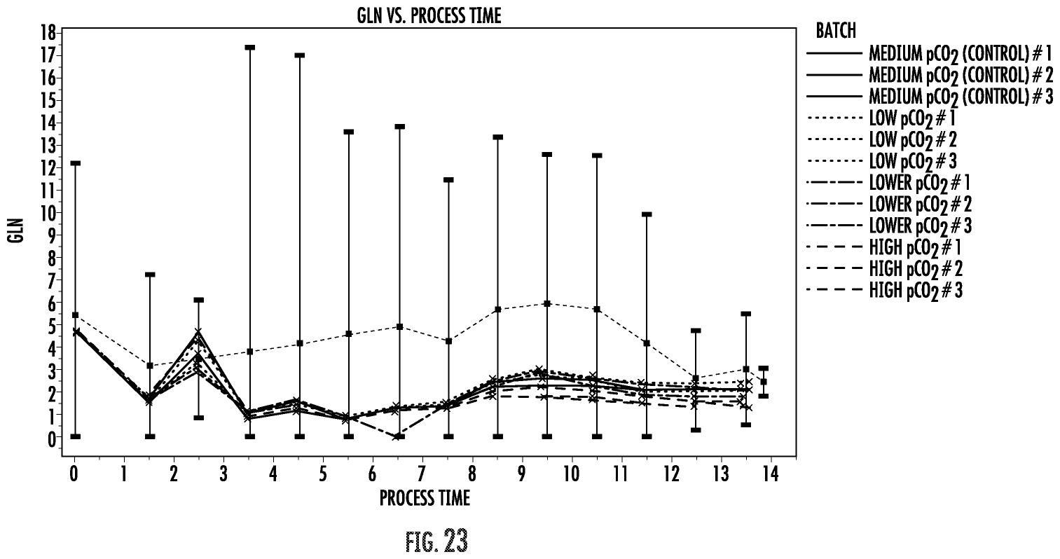

(a) viable cell density (VCD) (FIG. 12);
(b) viability values (FIG. 13);
(c) pH values—the pH changed from day 6.5 in accordance with the zonal approach shown in Table 6 (FIG. 14);
(d) pCO₂ values—the pCO₂ changed from day 6.5 in accordance with the zonal approach shown in Table 6 (FIG. 15)
(e) glucose values (FIG. 16);
(f) potassium values (FIG. 17);
(g) sodium values—the change in sodium values after day 7 was likely due to a sensor change and was not expected to influence study results (FIG. 18);
(h) osmolality values—the atypical value at day 10 is likely due to a sample error (FIG. 19);
(i) glutamate values—the atypical value at day 6.5 is likely due to a sample error (FIG. 20);
(j) lactate values (FIG. 21);
(k) ammonia values—ammonia values may have been influenced by pH (FIG. 22); and
(l) glutamine values (FIG. 23).
These data show similarity amongst cells propagated under different air sparging conditions. See Table 6.

### Example 6—Production in Culture Using CO₂ Sparging of a Human IgG4 Monoclonal Antibody that Binds the Interleukin 4 (IL-4) Receptor

The following study was conducted to evaluate the effect of culture pCO₂ on the charge variant profile of a human IgG4 monoclonal antibody that binds to the IL-4R alpha (a) subunit and thereby inhibits Interleukin 4 (IL-4) and Interleukin 13 (IL-13) signaling.

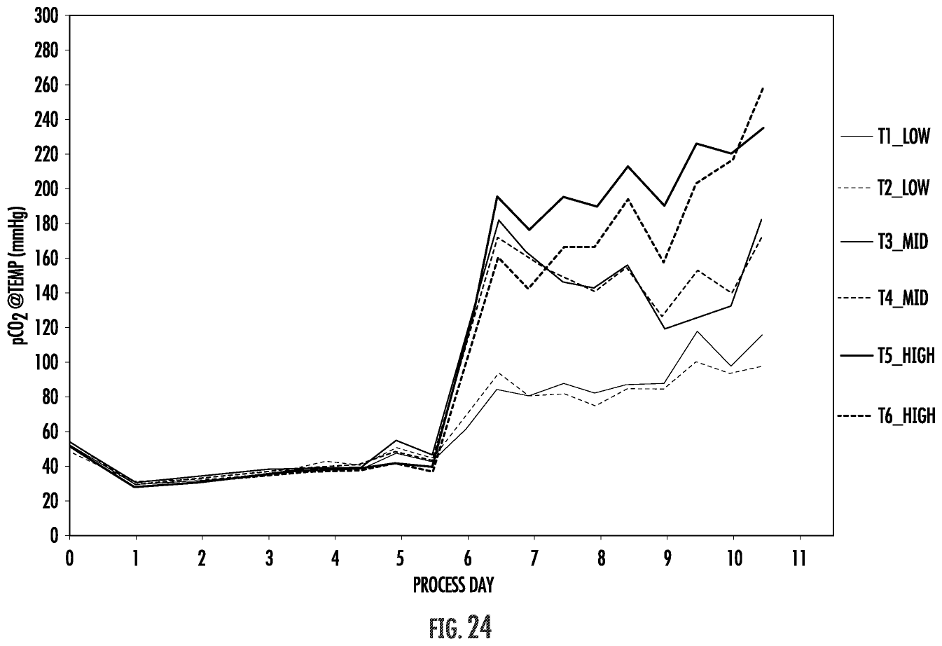

Culture media within a production bioreactor was inoculated with CHO cells at a concentration of about 12×10⁵ cells/ml, and allowed to grow in a fed-batch process. Once peak Viable Cell Density (VCD) of 200×10⁵ cells/mL was reached on Day 5.5, CO₂ sparging was modified as defined in Table 8 to vary pCO₂ levels within the cell culture. The resultant pCO₂ profiles of the three experimental conditions are provided in FIG. 24.

TABLE 8
Percentage Minimum CO₂ Sparge Flow Rate of Low and High pCO₂
Conditions Relative to Medium pCO₂ Condition (Control)

| Day | 0-5.5 | 5.5-6.0 | 6.0-10.5 |
|---|---|---|---|
| Low PCO₂ | 100 | 40 | 33 |
| Condition | | | |
| Medium PCO₂ | 100 | 100 | 100 |
| Condition | | | |
| High PCO₂ | 100 | 160 | 167 |
| Condition | | | |

Following 10.5 days of culture, the bioreactors where harvested and the monoclonal antibody was purified. The glycosylation and charge variant profiles were determined. It was noted that as pCO₂ levels within the production bioreactor increased, there was a concomitant decrease in levels of basic variants, as measured by imaged-capillary isoelectric focusing (iCIEF) (Table 9). In addition, an increase pCO₂ led to a concave acidic variant profile, peaking with mid pCO₂ condition, but dropping to the lowest percentage at the high pCO₂ condition (Table 10). The overall trend was a lower percentage of acidic charge variants, and the medium pCO₂ measure was likely a result of error.

TABLE 9
Effect of Culture pCO₂ on Basic Charge Variants

| Condition | Basic Variants (%) |
|---|---|
| Low pCO₂ | 9.0 |
| Medium pCO₂ | 8.3 |
| High pCO₂ | 7.9 |

TABLE 10
Effect of Culture pCO₂ on Acidic Charge Variants

| Condition | Acidic Variants (%) |
|---|---|
| Low pCO₂ | 37.0 |
| Medium pCO₂ | 39.0 |
| High pCO₂ | 36.0 |

The detailed statistical analyses in Example 5 above established that increased pCO₂, and not decreased pH, was the only statistically significant term for lowering the percentage of acidic charge variants (Region 1) with human IgG4 monoclonal antibodies. Thus, for the IgG class, represented by a human IgG4 monoclonal antibody here, increased pCO₂ itself lowers the percentage of acidic charge variants, and the lowering of the percentage of acidic charge variants in IgG4 antibodies is not caused by decreased pH values.

It is to be understood that the description, specific examples and data, while indicating exemplary embodiments, are given by way of illustration and are not intended to limit the present invention. Various changes and modifications within the present inventions, including combining embodiments in whole and in part, will become apparent to the skilled artisan from the discussion, disclosure and data contained herein, and thus are considered part of the inventions.

1. A method for reducing the percentage of acidic charge variants in antibody products produced by mammalian cells in culture, wherein the method comprises
seeding media with mammalian cells that produce antibodies; and
culturing the cells under pCO₂ conditions that allow the mammalian cells to produce antibody products with less acidic acid variants than would be obtained without the pCO₂ conditions, wherein the pCO₂ conditions are 120 mmHg to 140 mmHg of CO₂ in the media.

2. The method according to claim 1, wherein the pCO₂ conditions are attained by sparging.

3. The method according to claim 2, wherein the pCO₂ conditions are attained by CO₂ sparging.

4. The method according to claim 1, wherein the antibodies produced under the pCO₂ conditions have 0.5% to 4% less acidic variants than would be obtained without the pCO₂ conditions.

5. The method according to claim 1, wherein the antibodies are monoclonal antibodies.

6. The method according to claim 5, wherein the antibodies are capable of binding to PD-1 factor.

7. The method according to claim 5, wherein the antibodies are capable of binding IL-4 receptors.

8. The method according to claim 5, wherein the antibodies are human monoclonal antibodies.

9. The method according to claim 8, wherein the antibodies are human monoclonal antibodies are IgG antibodies.

10. The method according to claim 9, wherein the IgG antibodies are IgG4 antibodies.

11. The method according to of claim 1, wherein the cells are cultured for 10-15 days.

12. The method according to of claim 1, wherein the mammalian cells are CHO cells.

13. A method of controlling heterogeneity in antibodies produced by mammalian cells in culture, wherein the method comprises
seeding media with mammalian cells that produce antibodies; and
culturing the cells under pCO₂ conditions that allow the mammalian cells to produce antibodies, wherein the main peak form of antibodies produced by the cells comprises between 38% to 65% of total antibodies, the acidic variant of the antibodies comprises 20% to 47% of total antibodies and the basic variant of the antibodies comprises up to 36% of total antibodies.

14. The method according to claim 13, wherein the antibodies are monoclonal antibodies.

15. The method according to claim 14, wherein the monoclonal antibodies are capable of binding to PD-1 factor.

16. The method according to claim 14, wherein the antibodies are capable of binding IL-4 receptors.

17-19. (canceled)

20. A method of controlling heterogeneity in antibodies, antibody derivatives or antibody fragments produced by mammalian cells in culture, wherein the method comprises
seeding media with mammalian cells that produce antibodies, antibody derivatives or antibody fragments; and
culturing the cells under pCO₂ conditions that allow the mammalian cells to produce antibodies, antibody derivatives or antibody fragments, wherein the main peak form of antibodies, antibody derivatives or antibody fragments produced by the cells comprise between 50% to 70% of total antibodies, antibody derivatives or antibody fragments, the acidic variant of the antibodies, antibody derivatives or antibody fragments comprise 20% to 47% of total antibodies, antibody derivatives or antibody fragments and the basic variant of the antibodies, antibody derivatives or antibody fragments comprise up to 15% of total antibodies, antibody derivatives or antibody fragments.

21-27. (canceled)

28. The method according to claim 20, wherein the mammalian cells produce human monoclonal antibodies.

29. The method according to claim 28, wherein the human monoclonal antibodies are IgG1 antibodies.

30. The method according to claim 29, wherein the IgG antibodies are IgG4 antibodies.

31. The method according to claim 20, wherein the pCO₂ conditions are between 30 mmHg and 210 mmHg during the culturing.

32-37. (canceled)

38. An antibody product produced by the method according to claim 1.
